(19) United States
(12) Patent Application Publication
(10) Pub. No.: US 2023/0326599 A1
(43) Pub. Date: Oct. 12, 2023

(54) MEDICAL PLANNING ASSISTANCE SYSTEM, MEDICAL PLANNING ASSISTANCE METHOD, AND RECORDING MEDIUM HAVING STORED THEREIN MEDICAL PLANNING ASSISTANCE PROGRAM

(71) Applicant: NEC Corporation, Minato-ku, Tokyo (JP)

(72) Inventor: Ryosuke Togawa, Tokyo (JP)

(73) Assignee: NEC Corporation, Minato-ku, Tokyo (JP)

(21) Appl. No.: 18/022,349

(22) PCT Filed: Sep. 29, 2020
(86) PCT No.: PCT/JP2020/036753

§ 371 Date: Feb. 21, 2023

(51) Int. Cl. G16H 50/20; G16H 10/60; G16H 50/70

(52) U.S. Cl. CPC G16H 50/20; G16H 10/60; G16H 50/70; G16H 40/60

(57) ABSTRACT

A medical planning assistance system provides assistance to a medical worker in performing an appropriate medical practice according to the condition of a patient by being provided with: an acquisition unit that acquires patient condition information about the condition of a first patient; an inference model that is a model obtained by learning of a relation among a condition of a second patient, a medical practice performed on the second patient, and a condition change of the second patient due to the medical practice performed on the second patient; an inference unit that infers recommended medical practice to be recommended for the first patient, on the basis of the inference model and the patient condition information acquired by the acquisition unit; and an output unit that outputs the recommended medical practice inferred by the inference unit.

### TECHNICAL FIELD

The present invention relates to a medical planning assistance system, a medical planning assistance device, a medical planning assistance method, and a recording medium storing a medical planning assistance program.

### BACKGROUND ART

It is expected to assist people engaged in various fields including a medical field by using a rapidly developing machine learning technology.

PTL 1 discloses a medical data structure including medical care data for a patient including a plurality of elements, and morbidity information for specifying whether the patient is a person under a poor physical condition who has been hospitalized or a normal person who has not been hospitalized. This data structure is used in a process of executing supervised learning in which, in a case where the patient is a recovery patient, a weight of one of the elements of the medical care data is changed according to a weighting rule to change tensor data, and a core tensor is generated to be similar to a target core tensor.

In addition, PTL 2 discloses a learning program for generating, from training data, each piece of the training data including explanatory variables and an objective variable, a hypothesis set in which hypotheses are listed, each of the hypotheses being a combination of the explanatory variables and meeting a specific condition, so that one piece of the training data is classified into one of the hypotheses. This learning program performs learning for calculating a weight of each of the plurality of hypotheses on the basis of whether each piece of the training data satisfies each of the plurality of hypotheses included in the hypothesis set.

### Citation List

### Patent Literature

[PTL 1] JP 2020-101901 A

[PTL 2] JP 2020-046888 A

### Non Patent Literature

[NPL 1] Lu Wang, Wenchao Yu, Wei Wang, Wei Cheng, Martin

Renqiang Min, Bo Zong, Xiaofeng He, Hongyuan Zha, Wei Zhang, Haifeng Chen, “Adversarial Cooperative Imitation Learning for Dynamic Treatment Regimes”, WWW '20, Apr. 20-24, 2020, Taipei, Taiwan, Pages 1785-1795

### SUMMARY OF INVENTION

### Technical Problem

In the medical field, a medical practice for a patient who is in a certain condition is usually determined on the basis of knowledge and experience of an individual doctor. For this reason, a determination criterion in determining a medical practice is not sufficiently accurate, and for example, an inappropriate medical practice may be performed by a medical worker such as a doctor with insufficient knowledge and experience, and as a result, a condition of a patient may not be improved or may deteriorate. PTL 1 and PTL 2 do not mention this problem.

A main object of the present invention is to provide a medical planning assistance system and the like capable of assisting a medical worker to perform an appropriate medical practice according to a condition of a patient.

### Solution to Problem

A medical planning assistance system according to an aspect of the present invention includes: an acquisition means configured to acquire patient condition information regarding a condition of a first patient; an estimation means configured to estimate a recommended medical practice for the first patient based on the patient condition information acquired by the acquisition means and an estimation model; and an output means configured to output the recommended medical practice estimated by the estimation means, in which the estimation model is a model that has learned a relationship between a condition of a second patient, a medical practice performed on the second patient, and a change in condition of the second patient caused by the medical practice performed on the second patient.

From another viewpoint for achieving the above-described object, a medical planning assistance method performed by an information processing device according to an aspect of the present invention includes: acquiring patient condition information regarding a condition of a first patient; estimating a recommended medical practice for the first patient based on the acquired patient condition information and an estimation model; and outputting the estimated recommended medical practice, in which the estimation model is a model that has learned a relationship between a condition of the second patient, a medical practice performed on the second patient, and a change in condition of the second patient caused by the medical practice performed on the second patient.

From a further viewpoint for achieving the above-described object, a medical planning assistance program according to an aspect of the present invention causes a computer to execute: an acquisition process of acquiring patient condition information regarding a condition of a first patient; an estimation process of estimating a recommended medical practice for the first patient based on the patient condition information acquired by the acquisition process and an estimation model; and an output process of outputting the recommended medical practice estimated by the estimation process, in which the estimation model is a model that has learned a relationship between a condition of a second patient, a medical practice performed on the second patient, and a change in condition of the second patient caused by the medical practice performed on the second patient.

Furthermore, the present invention can also be implemented by a non-volatile computer-readable recording medium storing the medical planning assistance program (computer program).

### Advantageous Effects of Invention

According to the present invention, it is possible to obtain a medical planning assistance system and the like capable of assisting a medical worker to perform an appropriate medical practice according to a condition of a patient.

### BRIEF DESCRIPTION OF DRAWINGS

FIG. 1 is a block diagram illustrating a configuration of a medical planning assistance system 10 according to a first example embodiment of the present invention.

FIG. 2 is a diagram illustrating an example of patient condition information 101 according to the first example embodiment of the present invention.

FIG. 3 is a diagram illustrating an example of medical practice information 102 according to the first example embodiment of the present invention.

FIG. 4 is a diagram illustrating an example of condition change information 103 according to the first example embodiment of the present invention.

FIG. 5 is a flowchart illustrating an operation (process) of the medical planning assistance system 10 for generating an estimation model 140 (performing machine learning) according to the first example embodiment of the present invention.

FIG. 6 is a diagram illustrating an aspect in which an output unit 16 displays patient condition information 101 and medical practice information 102 for a patient who is under treatment, and an estimated recommended medical practice 104 on a display screen 200 according to the first example embodiment of the present invention.

FIG. 7 is a flowchart illustrating an operation of the medical planning assistance system 10 for estimating a recommended medical practice 104 according to the first example embodiment of the present invention.

FIG. 8 is a block diagram illustrating a configuration of a medical planning assistance system 40 according to a second example embodiment of the present invention.

FIG. 9 is a diagram illustrating a configuration of an information processing system 900 capable of implementing the medical planning assistance system 10 according to the first example embodiment of the present invention or the medical planning assistance system 30 according to the second example embodiment of the present invention.

### EXAMPLE EMBODIMENT

A system according to an example embodiment to be described below uses a trained model (also referred to as an estimation model) generated by machine learning (e.g., deep learning) when estimating a 10 target event from certain input information. Furthermore, the system may use conventional machine learning techniques such as adversarial cooperative imitation learning (ACIL) and skill acquisition learning (SAiL) disclosed in NPL 1.

ACIL is a technique for avoiding a failure by inputting failure cases, while applying adversarial imitation learning (AIL), which is a technique for imitating behaviors in success cases by inputting past success cases. SAiL is a technique for accurately imitating (learning) a behavior of a skilled person by providing a plurality of behavior imitating devices and selecting a behavior imitating device according to the situation. Note that, in the example embodiments to be described below, detailed description of these techniques will be omitted.

Hereinafter, example embodiments of the present invention will be described in detail with reference to the drawings.

### First Example Embodiment

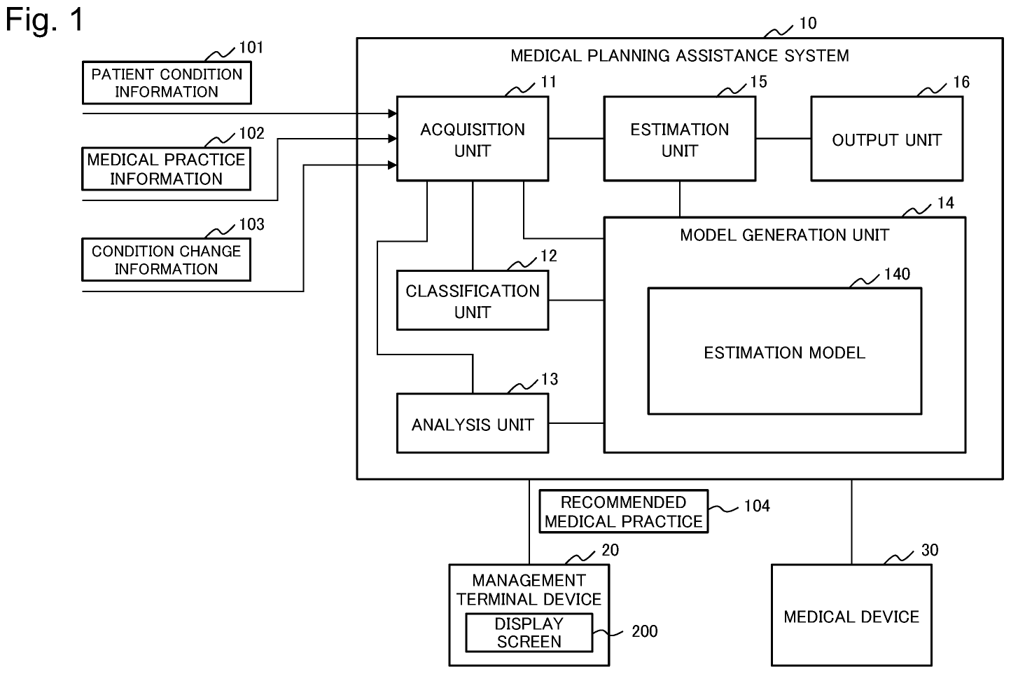

FIG. 1 is a block diagram illustrating a configuration of a medical planning assistance system 10 according to a first example embodiment of the present invention. The medical planning assistance system 10 is a system that assists a medical worker to determine an appropriate medical practice according to a condition of a patient. The medical worker includes a doctor who performs a medical practice, and a nurse, a physical therapist, an occupational therapist, a radiologist, and the like who support the doctor according to an instruction from the doctor.

The medical planning assistance system 10 generates a trained model (also referred to as an estimation model) using information about conditions of patients and medical practices performed on the patients in various treatment cases performed in the past, which are labeled as success cases in which conditions of patients have been improved or failure cases in which conditions of patients have not been improved. Using the trained model, the medical planning assistance system 10 estimates a medical practice recommended to be performed on a patient who is under treatment according to a condition of the patient. The medical planning assistance system 10 includes at least one information processing device.

A management terminal device 20 (also referred to as a display device) and a medical device 30 are communicably connected to the medical planning assistance system 10. The management terminal device 20 is, for example, a personal computer or another information processing device used when a medical worker who uses the medical planning assistance system 10 inputs information to the medical planning assistance system 10 or checks information output from the medical planning assistance system 10. The management terminal device 20 includes a display screen 200 that displays information output from the medical planning assistance system 10.

The medical device 30 is, for example, a device that measures a patient's condition such as a body temperature or a blood pressure, a device capable of adjusting an amount of medicine to be administered to a patient, or the like. The medical planning assistance system 10 can collect data indicating a condition of a patient from the medical device 30. Alternatively, the medical planning assistance system 10 can control an operation of the medical device 30 when a doctor performs a medical practice using the medical device 30.

The medical planning assistance system 10 includes an acquisition unit 11, a classification unit 12, an analysis unit 13, a model generation unit 14, an estimation unit 15, and an output unit 16. The acquisition unit 11, the classification unit 12, the analysis unit 13, the model generation unit 14, the estimation unit 15, and the output unit 16 are examples of an acquisition means, a classification means, an analysis means, a model generation means, an estimation means, and an output means, respectively.

Next, an operation of the medical planning assistance system 10 for generating or updating (re-training) an estimation model 140 for estimating a recommended medical practice 104, which is a medical practice recommended to be performed on a patient who is under treatment (hereinafter also referred to as a “subject patient”), according to the present example embodiment will be described. Thereafter, an operation for estimating a recommended medical practice 104 for the patient using the estimation model 140 will be described.

### <Operation for Generating (and Updating or Re-training) Estimation Model 140>

First, an operation of the medical planning assistance system 10 from acquiring information for treatment to generating or updating an estimation model 140 for estimating a medical practice recommended to be performed on a patient who is under treatment according to the present example embodiment will be described.

The acquisition unit 11 acquires patient condition information 101, medical practice information 102, and condition change information 103 in past treatment cases to be learned from a computer device or a database (not illustrated) via a network. For example, the acquisition unit 11 may periodically acquire patient condition information 101, medical practice information 102, and condition change information 103. For example, the acquisition unit 11 may acquire the patient condition information 101, the medical practice information 102, and the condition change information 103 according to an instruction input by a medical worker via the management terminal device 20.

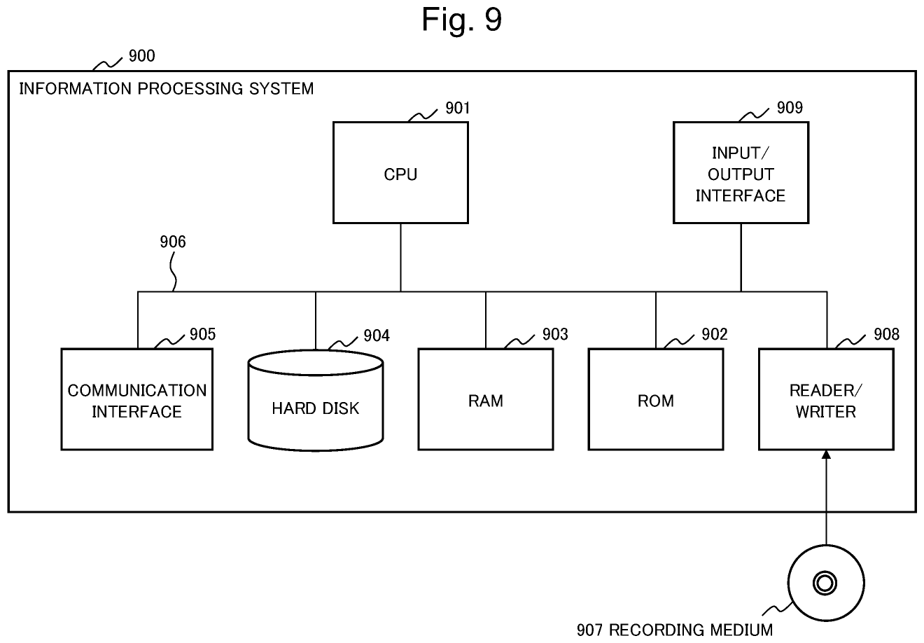

The acquisition unit 11 includes, for example, a communication circuit connected to one or more computer devices or databases that transmits the patient condition information 101, the medical practice information 102, and the condition change information 103, and a storage device that stores information acquired by the communication circuit. The storage device may be a hard disk 904 or a RAM 903 of an information processing system 900 illustrated in FIG. 9 to be described below.

The patient condition information 101 is information indicating a history of conditions for each patient. The condition of the patient is, for example, a body temperature, a heart rate, a blood pressure, a blood condition such as a blood oxygen concentration or an amount or concentration of a substance contained in the blood, a urine condition such as a color, an odor, or a containing substance of the urine, a feces condition, or the like. Alternatively, the condition of the patient is, for example, a consciousness condition such as a level of consciousness, a pain condition such as whether there is a pain or a degree of pain, a joint movement condition such as a joint-movable range, or the like obtained when a doctor diagnoses a patient. Alternatively, the condition of the patient is, for example, a condition of an organ evaluated by a doctor from an image obtained by a medical device such as an X-ray. Note that the patient condition information is not limited to the above-described information as long as the patient condition information is information regarding a health condition or a biological condition of a patient.

The above-described patient condition information may be acquired through a predetermined examination such as a medical examination, may be acquired at the time of medical examination or diagnosis, or may be acquired by a wearable device.

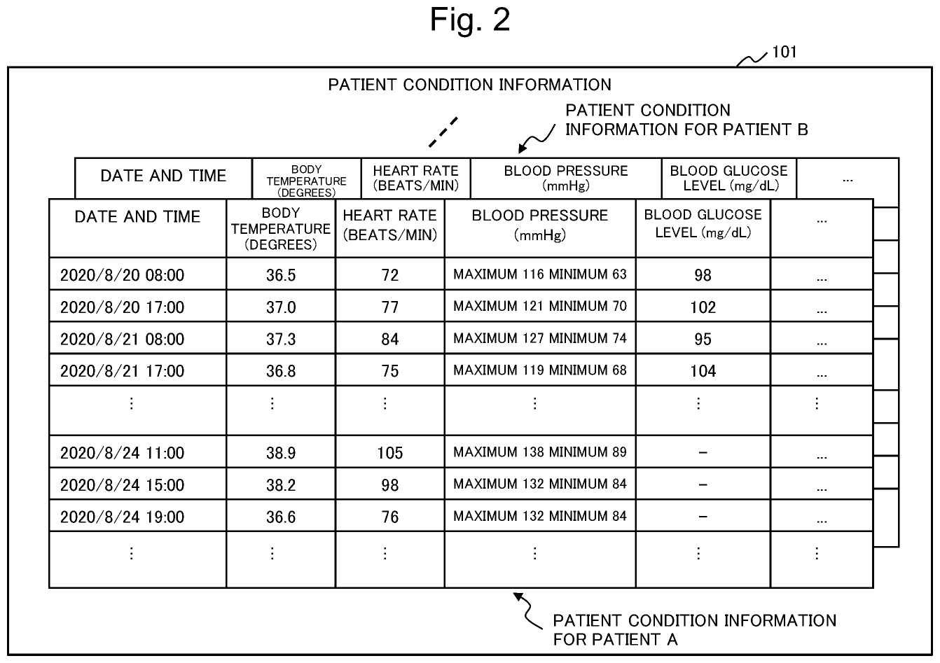

FIG. 2 is a diagram illustrating an example of data on the patient condition information 101 according to the present example embodiment. The patient condition information 101 indicates a history (progress) of patient's conditions for each patient such as patient A or patient B. The patient condition information 101 illustrated in FIG. 2 indicates a body temperature, a heart rate, a blood pressure, a blood glucose level, and the like as a condition of a patient, but the condition of the patient indicated by the patient condition information 101 is not limited thereto.

Furthermore, although the patient condition information 101 illustrated in FIG. 2 indicates a condition of a patient using a specific numerical value, the patient condition information 101 may indicate a condition of a patient, for example, by indicating whether the value for each item is a normal value or an abnormal value.

The medical practice information 102 is information indicating a history of medical practices performed on each patient. For example, the medical practice is surgery, medication treatment, radiation treatment, rehabilitation treatment, or the like. For example, in a case where the medical practice is surgery, the medical practice information 102 indicates a body site on which the surgery is performed, a surgery method, and the like. The surgery method is, for example, laparotomy, catheter surgery, or the like. For example, in a case where the medical practice is medication treatment, the medical practice information 102 indicates a type (name or the like) of medicine to be administered, an administration method, a dosage, and the like. For example, in a case where the medical practice is radiation treatment, the medical practice information 102 indicates a type of radiation to be used, such as X-ray, γ-ray, or electron beam, an irradiation location, an irradiation time, and the like. For example, in a case where the medical practice is rehabilitation treatment, the medical practice information 102 indicates a treated body site, a specific type of rehabilitation, and the like.

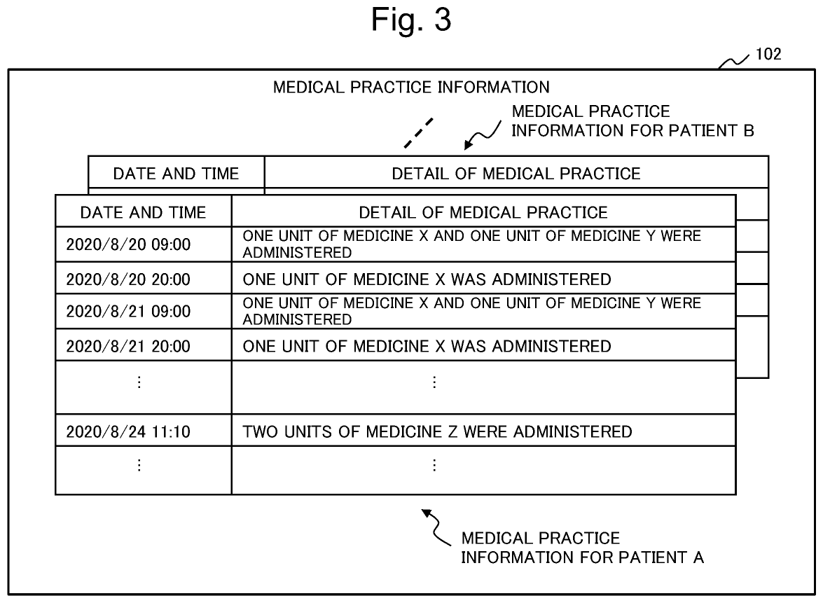

FIG. 3 is a diagram illustrating an example of the medical practice information 102 according to the present example embodiment. The medical practice information 102 indicates a history of medical practices performed for each patient such as patient A or patient B. The medical practice information 102 illustrated in FIG. 3 indicates that continuous medication treatment is performed on the patient A.

The condition change information 103 is information indicating a history of changes in condition for each patient. The change in condition indicates, for example, whether a condition of a patient has been improved or deteriorated, that is, whether a treatment has succeeded or failed. Whether the condition of the patient has been improved or deteriorated can be determined based on a determination criterion defined using a predetermined key performance indicator (KPI). In addition to whether the condition of the patient has been improved or deteriorated, the KPI may indicate that the condition of the patient remains unchanged, such as “the current condition being maintained”, in a case where the condition of the patient has not been improved or deteriorated by a predetermined value or more.

As the KPI, the above-described various indexes (e.g., body temperature, blood pressure, and blood glucose level) indicating a condition of a patient can be used. As an example of the determination criterion, each of the indexes may be used in such a manner that when the index increases to a predetermined reference value or more, this indicates that the condition has been deteriorated, and when the index decreases to the predetermined reference value or less, this indicates that the condition has been improved.

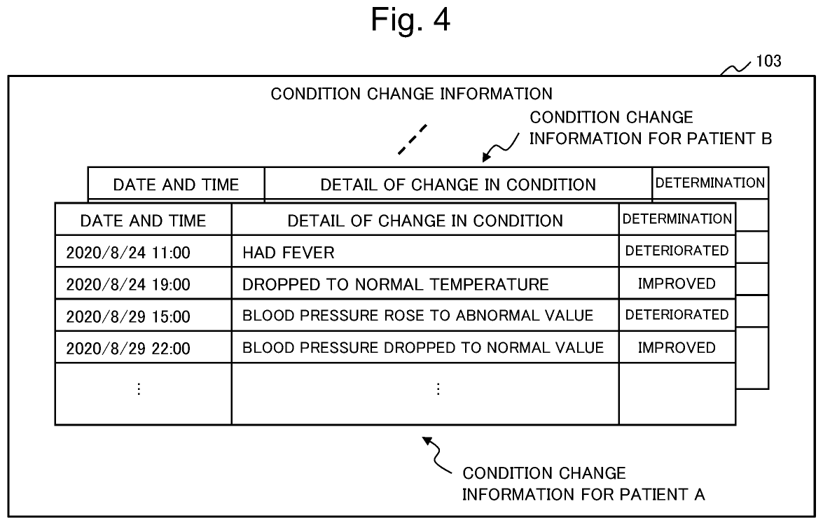

FIG. 4 is a diagram illustrating an example of the condition change information 103 according to the present example embodiment. The condition change information 103 indicates a history of whether conditions has been improved or deteriorated for each patient such as patient A or patient B. According to the condition change information 103 illustrated in FIG. 4, the patient A had a fever at 11:00 on Aug. 24, 2020, and the condition was deteriorated. For this reason, as indicated by the medical practice information 102 illustrated in FIG. 3, a medical practice of administering two units of medicine Z to the patient A is performed at 11:10 on Aug. 24, 2020. As a result, as indicated by the patient condition information 101 illustrated in FIG. 2 and the condition change information 103 illustrated in FIG. 4, the body temperature of the patient A drops to normal temperature at 19:00 on Aug. 24, 2020, and the condition was improved. The above-described examples illustrated in FIGS. 2 to 4 indicate that the treatment has succeeded by administering two units of the medicine Z to the patient A.

The acquisition unit 11 may generate the condition change information 103 from the patient condition information 101, rather than acquiring the condition change information 103 from the outside. The acquisition unit 11 can obtain a change in condition of a patient from a history of conditions for the patient indicated by the patient condition information 101. Then, the acquisition unit 11 may determine whether the condition of the patient has been improved or deteriorated based on the determination criterion defined using the above-described predetermined KPI given in advance.

The acquisition unit 11 stores the patient condition information 101, the medical practice information 102, and the condition change information 103 acquired as described above in a storage device (e.g., a memory or a hard disk) although not illustrated.

Using the patient condition information 101, the medical practice information 102, and the condition change information 103 acquired by the acquisition unit 11 as teacher data, the model generation unit 14 generates an estimation model 140 (a trained model) used when the estimation unit 15 to be described below estimates a recommended medical practice 104 by machine learning. The estimation model 140 is generated by learning a relationship between a condition of a past patient, a medical practice performed on the past patient, and a change in condition of the past patient caused by the medical practice. For example, the model generation unit 14 generates or updates an estimation model 140 by ACIL and SAiL, which are the above-described machine learning techniques. The model generation unit 14 performs machine learning for generating or updating an estimation model 140 using the teacher data described above through the processor.

A treatment case to be learned, which is indicated by the patient condition information 101, the medical practice information 102, and the condition change information 103, is labeled as a success case or a failure case depending on a result of determining whether a condition of a patient has been deteriorated or improved, which is indicated by the condition change information 103. As illustrated in FIGS. 2 to 4, the above-described treatment case where two units of the medicine Z were administered to the patient A who had a fever was a success case.

For example, in a treatment case where a condition of a patient has changed (improved or deteriorated) when a long period of time elapses after a certain medical practice, it is difficult to ascertain a causal relationship between the medical practice and the condition of the patient. With respect to such a treatment case, for example, expert-proven research data obtained by determining whether the case is a success case or a failure case from a long-term viewpoint can be used as teacher data.

When generating or updating the estimation model 140, the model generation unit 14 can use a past result of estimating a medical practice recommended for a patient by the estimation unit 15 to be described below with respect to teacher data.

For example, in the above-described example illustrated in FIGS. 2 and 3, when a patient who is under medication treatment with medicine X and medicine Y has a fever, the estimation unit 15 estimates a medical practice recommended for the patient. In this case, there is a result that the condition of the patient is improved by administering 2 units of the medicine Z as described above, but the estimation unit 15 estimates a recommended medical practice without referring to this result.

The model generation unit 14 determines whether the teacher data indicates a success case or a failure case based on the condition change information 103. When the teacher data indicates a success case, the model generation unit 14 generates or updates the estimation model 140 such that a result of estimating a recommended medical practice by the estimation unit 15 is closer to (similar to) an actually-performed medical practice indicated by the medical practice information 102.

For example, it is assumed that the estimation unit 15 estimates that one unit of the medicine Z is administered as a recommended medical practice with respect to the teacher data indicating a success case. In this case, the model generation unit 14 generates or updates the estimation model 140 so that the estimation unit 15 can estimate as a recommended medical practice that two units of the medicine Z are administered as actually performed. That is, the model generation unit 14 generates or updates the estimation model 140 so that the estimation unit 15 can estimate a medical practice closer (similar) to the medical practice performed in the success case.

Furthermore, when the teacher data indicates a failure case contrary to the above-described example, the model generation unit 14 generates or updates the estimation model 140 such that a result of estimating a recommended medical practice by the estimation unit 15 is farther away (different) from an actually-performed medical practice indicated by the medical practice information 102.

For example, as illustrated in FIGS. 2 to 4, it is assumed that, when a patient who was under medication treatment with the medicine X and the medicine Y had a fever, two units of the medicine Z were administered to the patient, but the condition of the patient was not improved. That is, the teacher data in this case indicates a failure case.

For example, it is assumed that the estimation unit 15 estimates that one unit of the medicine Z is administered as a recommended medical practice with respect to the teacher data indicating such a failure case.

In this case, the model generation unit 14 generates or updates the estimation model 140 so as not to estimate such a medical practice similar to the actually-performed administration of 2 units of the medicine Z as a recommended medical practice. That is, the model generation unit 14 generates or updates the estimation model 140 so that the estimation unit 15 estimates, for example, a medical practice of administering a medicine different from the medicine Z as a recommended medical practice.

The model generation unit 14 stores the estimation model 140 generated or updated as described above in a non-volatile storage device although not illustrated. The model generation unit 14 can gradually improve estimation precision by updating (also referred to as re-training) the estimation model 140, for example, every predetermined time interval.

The classification unit 12 illustrated in FIG. 1 classifies the medical practices indicated by the medical practice information 102 acquired by the acquisition unit 11 into groups based on similarity, and inputs details of the groups to the model generation unit 14. Meanwhile, it is assumed that a criterion for classification is given in advance to the classification unit 12, for example, by a medical worker or the like. For example, the classification unit 12 may input an identifier capable of identifying a group to the model generation unit 14.

For example, the classification unit 12 classifies a medical practice using a first medicine and a medical practice using a second medicine having the same effect as the first medicine while being different in type such as a name into the same group. More specifically, the first medicine is an original medicine and the second medicine is a generic medicine.

The classification unit 12 may classify medical practices relating to surgery, radiation treatment, and rehabilitation treatment, as well as such medication treatment, into groups based on similarity.

The classification unit 12 may also classify the medical practices into groups having hierarchical structures based on based on similarity. For example, the classification unit 12 classifies medical practices performed on cancer patients into large-scale grading groups, such as surgery, medication treatment, and radiation treatment. Then, the classification unit 12 classifies the medical practices into smaller-scale grading groups belonging to a large-scale group, for example, based on a surgery method, a type of medicine to be used, a type of radiation to be used, or the like.

In a case where the above-described groups are input from the classification unit 12, the model generation unit 14 generates or updates the estimation model 140 using the groups as medical practices. In this case, the estimation unit 15 estimates a medical practice group as a recommended medical practice based on the estimation model 140 generated using the medical practice groups.

The analysis unit 13 illustrated in FIG. 1 analyzes whether the patient condition information 101, the medical practice information 102, and the condition change information 103 acquired by the acquisition unit 11 indicate that a combination of a plurality of medical practices has affected a condition of a patient. For example, in a case where a plurality of medical practices are performed on a patient at the same timing, and as a result, it is recognized that the condition of the patient has been improved or deteriorated, the analysis unit 13 determines that the combination of medical practices has affected the condition of the patient. It is assumed that a criterion for determining that a combination of a plurality of medical practices has affected a condition of a patient is given in advance to the analysis unit 13, for example, by a medical worker or the like.

The combination of the plurality of medical practices refers to, for example, medication treatment with a combination of a plurality of medicines. Alternatively, the combination of the plurality of medical practices may be a combination of two or more medical practices, for example, among surgery, medication treatment, radiation treatment, and rehabilitation treatment.

When a certain combination of medical practices is found to have affected a condition of a patient as a result of the above-described analysis, the analysis unit 13 inputs the combination to the model generation unit 14. The combination is data related to the combined medical practices.

When the above-described combination is input from the analysis unit 13, the model generation unit 14 generates or updates the estimation model 140 using the combination as a medical practice. In this case, the estimation unit 15 estimates a combination of medical practices as a recommended medical practice based on the estimation model 140 generated using the combinations of medical practices.

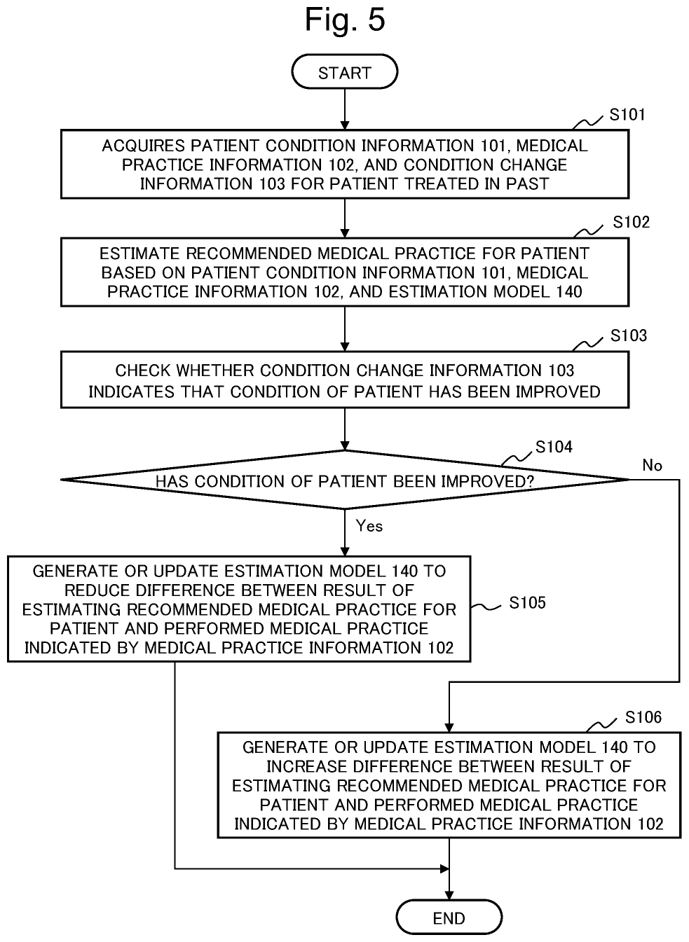

Next, an operation (process) of generating an estimation model 140 (performing machine learning) by the medical planning assistance system 10 according to the present example embodiment will be described in detail with reference to a flowchart of FIG. 5.

The acquisition unit 11 acquires patient condition information 101, medical practice information 102, and condition change information 103 for a patient treated in the past (step S101). The estimation unit 15 estimates a recommended medical practice for the patient on the basis of the patient condition information 101, the medical practice information 102 for the patient acquired by the acquisition unit 11, and the estimation model 140 (step S102).

The model generation unit 14 checks whether the condition change information 103 indicates that the condition of the patient has been improved (that is, whether the input treatment case is a success case or a failure case) (step S103).

When the condition change information 103 indicates that the condition of the patient has been improved (that is, the input treatment case is a success case) (Yes in step S104), the process proceeds to step S105. The model generation unit 14 generates or updates the estimation model 140 to reduce a difference between a result of estimating a recommended medical practice for the patient and a performed medical practice indicated by the medical practice information 102 (step S105), and the entire process ends.

When the condition change information 103 indicates that the condition of the patient has not been improved (that is, the input treatment case is a failure case) (Yes in step S104), the process proceeds to step S106. The model generation unit 14 generates or updates the estimation model 140 to increase a difference between a result of estimating a recommended medical practice for the patient and a performed medical practice indicated by the medical practice information 102 (step S106), and the entire process ends.

### <Operation for Estimating Medical Practice Recommended to Be Performed on Patient Who is Under Treatment>

Next, an operation of the medical planning assistance system 10 for estimating a medical practice recommended to be performed on a patient who is under treatment using the generated or updated estimation model 140 according to the present example embodiment will be described.

The acquisition unit 11 acquires patient condition information 101 and medical practice information 102 from an external device (not illustrated), in the same manner as when the medical planning assistance system 10 generates the estimation model 140. However, the acquisition unit 11 acquires these information not as the above-described teacher data but as data to be estimated regarding a medical practice recommended to be performed on a patient who is under treatment (also referred to as a first patient).

In addition, as described above, it is assumed that an estimation model 140 is generated based on patient condition information 101, medical practice information 102, and condition change information 103 for a patient treated in the past (also referred to as a second patient). In this case, the acquisition unit 11 acquires patient condition information 101 and medical practice information 102 for a patient who is under treatment, which are input, for example, by a medical worker such as a doctor via the management terminal device 20. Aspects of the patient condition information 101 and the medical practice information 102 for the patient who is under treatment are similar to those of the patient condition information 101 and the medical practice information 102 used to generate the estimation model 140 illustrated in FIGS. 2 and 3.

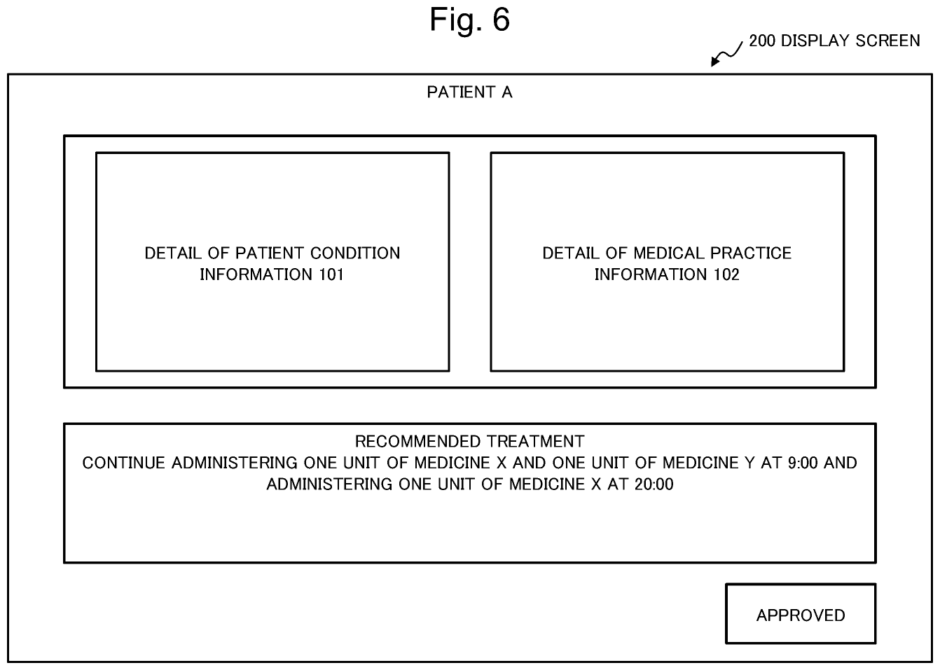

FIG. 6 is a diagram illustrating an aspect in which the output unit 16 displays patient condition information 101 and medical practice information 102 for a patient who is under treatment, and a recommended medical practice 104 estimated by the estimation unit 15 on the display screen 200 according to the present example embodiment.

The display screen 200 illustrated in FIG. 6 is displayed by the output unit 16 after a doctor among medical workers logs in the medical planning assistance system 10 with his/her identification (ID) via the management terminal device 20. The output unit 16 displays the display screen 200 illustrated in FIG. 6 according to a doctor's input operation for selecting the patient A on a screen for selecting one of a plurality of patients although not illustrated.

The output unit 16 outputs at least one of patient condition information 101, medical practice information 102, and condition change information 103 for a subject patient.

For example, as illustrated in FIG. 6, the output unit 16 serving as a display control unit displays the patient condition information 101 and the medical practice information 102 for the patient A on the display screen 200. The patient condition information 101 and the medical practice information 102 may include information collected from the medical device 30, information separately input by a medical worker such as a nurse via the management terminal device 20, or the like in addition to the information input by the doctor's input operation on the display screen 200 illustrated in FIG. 6.

The patient condition information 101 and the medical practice information 102 for the patient A who is under treatment on the display screen 200 illustrated in FIG. 6, which are input by the doctor, are stored in a computer device or a database in which the patient condition information 101 and the medical practice information 102 are managed by the medical planning assistance system 10.

The estimation unit 15 estimates a recommended medical practice 104 that is a medical practice recommended to be performed on the patient who is under treatment based on the patient condition information 101, the medical practice information 102 for the patient under treatment acquired by the acquisition unit 11, and the estimation model 140. Furthermore, the estimation unit 15 functions to estimate a recommended medical practice based on the patient condition information 101, the medical practice information 102 input as teacher data, and the estimation model 140.

In a case where the estimation model 140 is generated using groups of medical practices by the above-described operation of the classification unit 12, the estimation unit 15 may estimate a group of medical practices as a recommended medical practice 104.

In a case where the estimation model 140 is generated using combinations of medical practices by the above-described operation of the analysis unit 13, the estimation unit 15 may estimate a combination of medical practices as a recommended medical practice 104. For example, the recommended medical practice 104 as a combination of medical practices is a series of medical practices including a plurality of medical practices such as “catheter surgery is performed, and then medicine X is administered by drip infusion twice a day, morning and night, for a week”.

The estimation unit 15 outputs the estimated recommended medical practice 104 to the output unit 16. At this time, the estimation unit 15 may also output a reason why the recommended medical practice 104 is estimated to the output unit 16. The estimation unit 15 can output the reason for the estimation with an attention technology in deep learning.

The output unit 16 outputs the recommended medical practice estimated by the estimation unit 15. For example, the output unit 16 serving as a display control unit displays the recommended medical practice 104 input from the estimation unit 15 on the display screen 200 in the management terminal device 20. That is, the output unit 16 controls the management terminal device 20 to display the recommended medical practice 104 on the display screen 200 of the management terminal device 20. At a timing when the doctor completes an input operation of inputting the patient condition information 101 and the medical practice information 102 for the patient A on the display screen 200, the output unit 16 may display the recommended medical practice 104 as feedback on the input operation on the display screen 200.

In the example illustrated in FIG. 6, the estimation unit 15 estimates a recommended medical practice 104 indicating “continue administering one unit of medicine X and one unit of medicine Y at 9:00 and administering one unit of medicine X at 20:00” for the patient A who is under medication treatment with the medicine X and the medicine Y. Alternatively, the estimation unit 15 estimates a recommended medical practice 104 such as “perform rehabilitation treatment for stabilizing a lower body trunk and increasing a motion range of dorsiflexion for an ankle joint”, for example, for the patient B who is under rehabilitation treatment.

Furthermore, as illustrated in FIG. 6, the output unit 16 displays an “approved” button on the display screen 200 in order to prompt the doctor to perform an input operation indicating whether to approve the recommended medical practice 104 displayed on the display screen 200.

For example, when the “approved” button is selected by the doctor through a mouse operation or a touch operation, the output unit 16 generates control information for the medical device 30 to be used when the doctor performs the recommended medical practice 104 displayed on the display screen 200, and transmits the generated control information to the medical device 30.

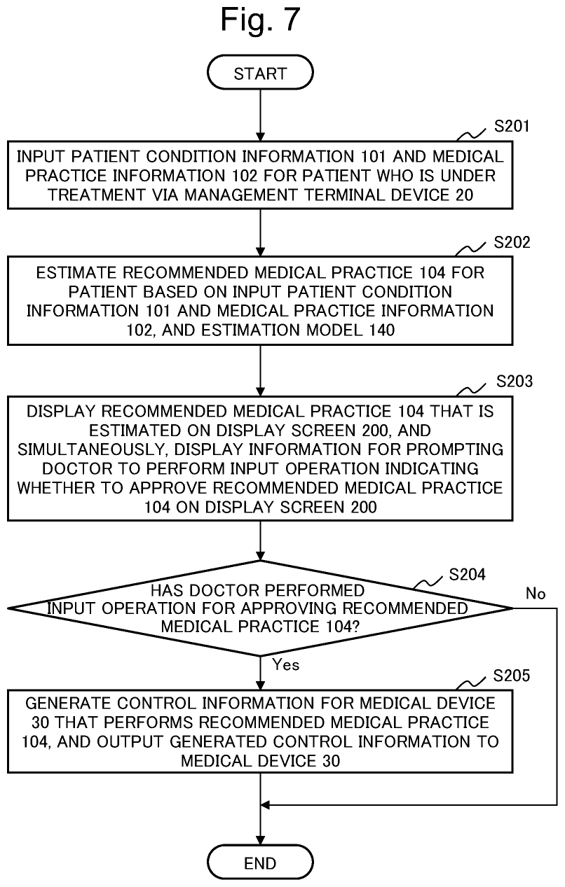

Next, an operation (process) of the medical planning assistance system 10 for estimating a recommended medical practice 104 according to the present example embodiment will be described in detail with reference to a flowchart of FIG. 7.

Patient condition information 101 and medical practice information 102 for a patient who is under treatment are input via the management terminal device 20 (step S201). The estimation unit 15 estimates a recommended medical practice 104 for the patient based on the input patient condition information 101, the medical practice information 102, and the estimation model 140 (step S202).

The output unit 16 displays the recommended medical practice 104 estimated by the estimation unit 15 on the display screen 200, and simultaneously, displays information for prompting a doctor to perform an input operation indicating whether to approve the recommended medical practice 104 on the display screen 200 (step S203).

When the doctor does not perform an input operation for approving the recommended medical practice 104 on the display screen 200 (No in step S204), the entire process ends. When the doctor performs an input operation for approving the recommended medical practice 104 on the display screen 200 (Yes in step S204), the output unit 16 generates control information for the medical device 30 that performs the recommended medical practice 104 and outputs the generated control information to the medical device 30, and the entire process ends.

The medical planning assistance system 10 according to the present example embodiment can assist a medical worker to perform an appropriate medical practice according to a condition of a patient. This is because the medical planning assistance system 10 estimates a medical practice recommended for a patient who is currently being treated based on a condition of the patient who is currently being treated and the estimation model 140 generated from past treatment cases.

Hereinafter, an effect achieved by the medical planning assistance system 10 according to the present example embodiment will be described in detail.

In the medical field, a medical practice for a patient who is in a certain condition is usually determined on the basis of knowledge and experience of an individual doctor. For this reason, a determination criterion in determining a medical practice is not sufficiently accurate, and for example, an inappropriate medical practice may be performed by a medical worker such as a doctor with insufficient knowledge and experience, and as a result, a condition of a patient may not be improved or may deteriorate.

To solve such a problem, the medical planning assistance system 10 according to the present example embodiment includes an acquisition unit 11, an estimation model 140, an estimation unit 15, and an output unit 16, and operates, for example, as described above with reference to FIGS. 1 to 7. That is, the acquisition unit 11 acquires patient condition information 101 regarding a condition of a patient (a first patient) who is under treatment. The estimation model 140 is a model that has learned a relationship between a condition of a past patient (a second patient), a medical practice performed on the past patient, and a change in condition of the past patient caused by the medical practice performed on the past patient. Then, the estimation unit 15 estimates a recommended medical practice 104 for the patient who is under treatment based on the patient condition information 101 regarding the condition of the patient who is under treatment and the estimation model 140. As a result, the medical planning assistance system 10 can provide suitable assistance in treating a patient, for example, to prevent a medical worker such as a doctor with insufficient knowledge and experience from performing an inappropriate medical practice.

Furthermore, the medical planning assistance system 10 according to the present example embodiment includes a model generation unit 14 that generates or updates the estimation model 140 based on patient condition information 101, medical practice information 102, and condition change information 103 for the past patient. In a process of generating or updating the estimation model 140, in the medical planning assistance system 10, a medical practice recommended for the past patient is estimated by the estimation unit 15 based on the patient condition information 101, the medical practice information 102 for the past patient, and the estimation model 140. Then, the model generation unit 14 updates the estimation model 140 to reduce a difference of the medical practice estimated by the estimation unit 15 from a medical practice actually performed in a treatment success case. Alternatively, the model generation unit 14 updates the estimation model 140 to increase a difference of the medical practice estimated by the estimation unit 15 from a medical practice actually performed in a treatment failure case. That is, since the medical planning assistance system 10 updates the estimation model 140 using both the treatment success case and the treatment failure case as teacher data, it is possible to construct the estimation model 140 capable of highly accurate estimation in a short period of time.

In addition, in a case where a recommended medical practice is estimated using the estimation model 140, there is a problem as follows. There are a plurality of medical practices having the same effect (that is, a similar effect), for example, medicines having the same effect while being different in type (name or the like), such as an original medicine and a generic medicine. In a case where such a plurality of medical practices are treated separately, the number of samples for each medical practice decreases, which causes a decrease in accuracy as teacher data. To solve such a problem, the medical planning assistance system 10 according to the present example embodiment includes a classification unit 12 that classifies medical practices into groups based on similarity and inputs the groups to the model generation unit 14, so that processing is performed in units of groups when the estimation model 140 is generated and the recommended medical practice 104 is estimated. As a result, the medical planning assistance system 10 can solve the above-described problem that the accuracy as teacher data decreases.

In addition, in a case where a recommended medical practice is estimated using the estimation model 140, there is another problem as follows. For example, in rehabilitation treatment or the like, a therapeutic effect may be obtained by performing a plurality of medical practices (medical procedures) in combination. Alternatively, a side effect may be caused by a combination of a plurality of medicines. Therefore, in a case where only a single medical practice is focused on, an effect of the combination of the plurality of medical practices described above cannot be grasped, which causes a decrease in accuracy as teacher data. To solve such a problem, the medical planning assistance system 10 according to the present example embodiment includes an analysis unit 13 that analyzes whether a combination of a plurality of medical practices has affected a condition of a patient and inputs the combination of medical practices that has affected the condition of the patient to the model generation unit 14, so that processing is performed in units of combinations when the estimation model 140 is generated and the recommended medical practice 104 is estimated. As a result, the medical planning assistance system 10 can solve the above-described problem that the accuracy as teacher data decreases.

In addition, when an input operation is performed on the display screen 200 by inputting a condition of a patient who is under treatment or a performed medical practice, the medical planning assistance system 10 according to the present example embodiment displays a recommended medical practice 104 as feedback thereon. As a result, the medical planning assistance system 10 can improve work efficiency for a medical worker such as a doctor.

In addition, the medical planning assistance system 10 according to the present example embodiment displays, on the display screen 200, an “approved” button for prompting a doctor to perform an input operation indicating whether to approve the recommended medical practice 104 on the display screen 200. Then, when the recommended medical practice 104 is approved by the doctor, the medical planning assistance system 10 generates control information for the medical device 30 that performs the recommended medical practice 104, and outputs the control information to the medical device 30. As a result, the medical planning assistance system 10 can prevent an inappropriate medical practice that has not been approved by the doctor from being erroneously performed.

### Second Example Embodiment

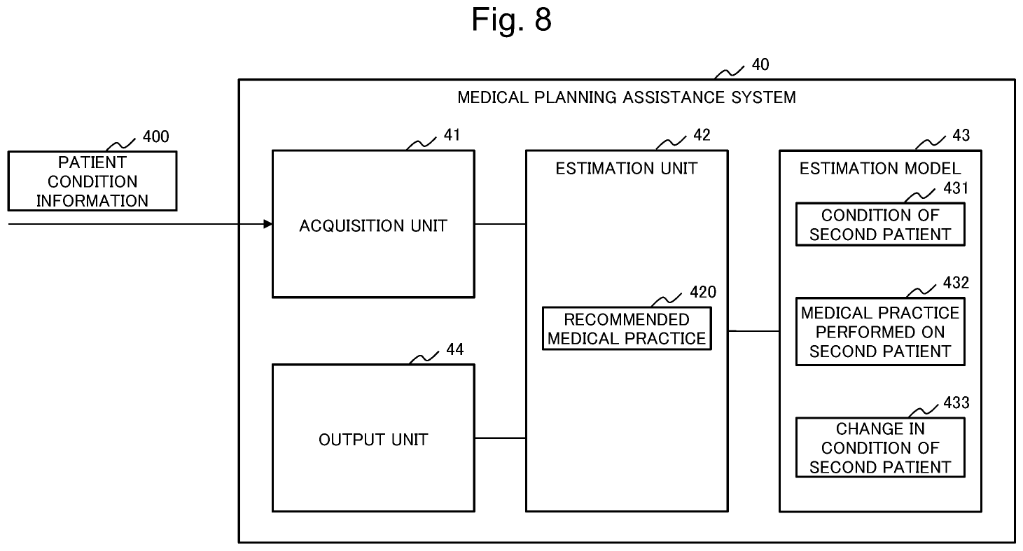

FIG. 8 is a block diagram illustrating a configuration of a medical planning assistance system 40 according to a second example embodiment of the present invention. The medical planning assistance system 40 includes an acquisition unit 41, an estimation unit 42 using an estimation model 43, and an output unit 44. The acquisition unit 41, the estimation unit 42, and the output unit 44 are examples of an acquisition means, an estimation means, and an output means, respectively.

The acquisition unit 41 acquires patient condition information 400 regarding a condition of a first patient. The acquisition unit 41 operates, for example, similarly to the acquisition unit 11 according to the first example embodiment. The first patient is, for example, a patient who currently being treated (that is, a subject patient), similarly to that in the first example embodiment.

The estimation model 43 is a model generated by learning a relationship between a condition 431 of a second patient, a medical practice 432 performed on the second patient, and a change 433 in condition of the second patient caused by the medical practice 432 performed on the second patient. The second patient is, for example, a past patient, similarly to that in the first example embodiment. For example, similarly to the estimation model 140 according to the first example embodiment, the estimation model 43 is a trained model indicating a result of performing machine learning about the relationship between the condition 431 of the second patient, the medical practice 432 performed on the second patient, and the change 433 in condition of the second patient caused by the medical practice 432 performed on the second patient.

The condition 431 of the second patient may be, for example, information similar to the patient condition information 101 described with reference to FIG. 2 in the first example embodiment. The medical practice 432 performed on the second patient may be, for example, information similar to the medical practice information 102 described with reference to FIG. 3 in the first example embodiment. The change 433 in condition of the second patient may be, for example, information similar to the condition change information 103 described with reference to FIG. 4 in the first example embodiment.

The estimation unit 42 estimates a recommended medical practice 420 for the first patient based on the patient condition information 400 acquired by the acquisition unit 41 and the estimation model 43. More specifically, the estimation unit 42 estimates a recommended medical practice 420, for example, by operating similarly to the estimation unit 15 according to the first example embodiment.

The output unit 44 outputs the recommended medical practice 420 estimated by the estimation unit 42. The output unit 44 operates, for example, similarly to the output unit 16 according to the first example embodiment.

The medical planning assistance system 40 according to the present example embodiment can assist a medical worker to perform an appropriate medical practice according to a condition of a patient. This is because the medical planning assistance system 40 estimates a medical practice recommended for a patient who is currently being treated based on a condition of the patient who is currently being treated and the estimation model 43 generated from past treatment cases.

### Example of Hardware Configuration

Each unit in the medical planning assistance system 10 illustrated in FIG. 1 or the medical planning assistance system 40 illustrated in FIG. 8 according to each of the above-described example embodiments can be achieved by dedicated hardware (HW) (electronic circuit). In addition, in FIGS. 1 and 8, at least the following components can be regarded as functional (processing) units (software modules) of a software program:
the acquisition units 11 and 41;
the classification unit 12;
the analysis unit 13;
the model generation unit 14;
the estimation units 15 and 42; and
the output units 16 and 44.

However, these units illustrated in the drawings are demarcated as components for convenience of description, and various configurations can be considered for implementation. An example of a hardware environment in this case will be described with reference to FIG. 9.

FIG. 9 is a diagram illustrating an example of a configuration of an information processing system 900 (computer system) capable of implementing the medical planning assistance system 10 according to the first example embodiment of the present invention or the medical planning assistance system 40 according to the second example embodiment of the present invention. That is, FIG. 9 illustrates a hardware environment capable of implementing each function in the above-described example embodiments as a configuration of at least one computer (information processing device) capable of implementing the medical planning assistance systems 10 and 40 illustrated in FIGS. 1 and 8.

The information processing system 900 illustrated in FIG. 9 includes the following components, but some of the following components may not be included therein:
a central processing unit (CPU) 901;
a read only memory (ROM) 902;
a random access memory (RAM) 903;
a hard disk (storage device) 904;
a communication interface 905 for communication with an external device;
a bus 906 (communication line);
a reader/writer 908 capable of reading and writing data stored in a recording medium 907 such as a compact disc read only memory (CD-ROM); and
an input/output interface 909 such as a monitor, a speaker, or a keyboard

That is, the information processing system 900 including the above-described components is a general computer in which the components are connected to each other via the bus 906. The information processing system 900 may include a plurality of CPUs 901, or may include a CPU 901 configured by multiple cores. The information processing system 900 may include a graphical processing unit (GPU) (not illustrated) in addition to the CPU 901.

In addition, the present invention described using the above-described example embodiments provides a computer program capable of implementing the following functions to the information processing system 900 illustrated in FIG. 9. The functions are functions of the above-described components in the block diagrams (FIGS. 1 and 8) referred to in the description of the example embodiments or the flowcharts (FIGS. 5 and 7). The present invention is achieved by reading the computer program to the CPU 901 of the hardware, and interpreting and executing the computer program. The computer program provided in the device may be stored in a readable/writable volatile memory (the RAM 903) or a non-volatile storage device such as the ROM 902 or the hard disk 904.

Furthermore, in the above-described case, a general procedure can be adopted currently as a method of providing the computer program in the hardware. Examples of the procedure include installing the program in the device via any type of recording medium 907 such as a CD-ROM and downloading the program from the outside via a communication line such as the Internet. In such a case, the present invention can be considered as being achieved by codes constituting the computer program or the recording medium 907 storing the codes.

The present invention has been described above using the above-described example embodiments as preferred embodiments. However, the present invention is not limited to the above-described example embodiments. That is, various aspects of the present invention that can be understood by those of ordinary skill in the art may be applied within the scope of the present invention.

Some or all of the above-described example embodiments may also be described as in the following supplementary notes. However, the present invention exemplarily described by the above-described example embodiments is not limited to the following supplementary notes.

### (Supplementary Note 1)

A medical planning assistance system including:
an acquisition means configured to acquire patient condition information regarding a condition of a first patient;
an estimation means configured to estimate a recommended medical practice for the first patient based on the patient condition information acquired by the acquisition means and an estimation model; and
an output means configured to output the recommended medical practice estimated by the estimation means,
in which the estimation model is a model that has learned a relationship between a condition of a second patient, a medical practice performed on the second patient, and a change in condition of the second patient caused by the medical practice performed on the second patient.

### (Supplementary Note 2)

The medical planning assistance system according to supplementary note 1,
in which the patient condition information for the first patient includes a history of biological conditions for the first patient and a history of medical practices performed on the first patient.

### (Supplementary Note 3)

The medical planning assistance system according to supplementary note 1 or 2,
in which the condition of each of the first and second patients includes at least one of a body temperature, a heart rate, a blood pressure, a blood condition, a urine condition, a feces condition, an organ condition, a consciousness condition, a pain condition, and a joint movement condition.

### (Supplementary Note 4)

The medical planning assistance system according to any one of supplementary notes 1 to 3,
in which the recommended medical practice and the medical practice performed on the first and second patients include at least one of surgery, medication treatment, radiation treatment, and rehabilitation treatment.

### (Supplementary Note 5)

The medical planning assistance system according to supplementary note 4,
in which the surgery includes a body site on which the surgery is to be performed and a surgery method,
the medication treatment includes at least one of a type of medicine to be administered, an administration method, and a dosage,
the radiation treatment includes at least one of a type of radiation to be used, an irradiation location, and an irradiation time, and
the rehabilitation treatment includes at least one of a body site to be treated and a type of rehabilitation.

### (Supplementary Note 6)

The medical planning assistance system according to any one of supplementary notes 1 to 5, further including
a model generation means configured to generate or update the estimation model based on the condition of the second patient, the medical practice performed on the second patient, and the change in condition of the second patient caused by the medical practice performed on the second patient.

### (Supplementary Note 7)

The medical planning assistance system according to supplementary note 6,
in which the estimation means estimates a medical practice recommended for the second patient based on the condition of the second patient, the medical practice performed on the second patient, and the estimation model,
when the change in condition of the second patient indicates that the condition of the second patient has been improved, the model generation means updates the estimation model to reduce a difference between the medical practice recommended for the second patient and the medical practice performed on the second patient, and
when the change in condition of the second patient indicates that the condition of the second patient has not been improved, the model generation means updates the estimation model to increase a difference between the medical practice recommended for the second patient and the medical practice performed on the second patient.

### (Supplementary Note 8)

The medical planning assistance system according to supplementary note 6 or 7, further including
a classification means configured to classify the medical practice performed on the second patient into a group based on similarity, and input the group to the model generation means,
in which the model generation means generates or updates the estimation model based on the group and the change in condition of the second patient caused by the medical practice performed on the second patient, and
the estimation means estimates the group to which the recommended medical practice belongs.

### (Supplementary Note 9)

The medical planning assistance system according to supplementary note 8,
in which the classification means classifies the medical practice performed on the second patient into the group having a hierarchical structure based on the similarity.

### (Supplementary Note 10)

The medical planning assistance system according to supplementary note 8 or 9,
in which the classification means classifies a first medical practice using a first medicine and a second medical practice using a second medicine into the same group, the second medicine having an equivalent effect to the first medicine while being different in type from the first medicine.

### (Supplementary Note 11)

The medical planning assistance system according to supplementary note 10,
in which the first medicine is an original medicine, and the second medicine is a generic medicine of the first medicine.

### (Supplementary Note 12)

The medical planning assistance system according to any one of supplementary notes 6 to 11, further including
an analysis means configured to analyze whether a combination of a plurality of medical practices performed on the second patient has affected the condition of the second patient, and input the combination of medical practices to the model generation means when the combination of medical practices has affected the condition of the second patient,
in which the model generation means uses the combination of medical practices as the medical practice performed on the second patient, and
the estimation means estimates the combination recommended for the first patient.

### (Supplementary Note 13)

The medical planning assistance system according to any one of supplementary notes 1 to 12,
in which the output means controls a display device to display the recommended medical practice when an input operation is performed to input the patient condition information for the first patient to the estimation means.

### (Supplementary Note 14)

The medical planning assistance system according to supplementary note 13,
in which the output means controls the display device to display information for prompting a doctor to perform an input operation indicating whether to approve the recommended medical practice.

### (Supplementary Note 15)

The medical planning assistance system according to supplementary note 14,
in which when the input operation performed by the doctor indicates that the recommended medical practice has been approved, the output means generates control information for a medical device that performs the recommended medical practice, and outputs the generated control information to the medical device

### (Supplementary Note 16)

A medical planning assistance device including:
an acquisition means configured to acquire patient condition information regarding a condition of a first patient;
an estimation means configured to estimate a recommended medical practice for the first patient based on the patient condition information acquired by the acquisition means and an estimation model; and
an output means configured to output the recommended medical practice estimated by the estimation means,
in which the estimation model is a model that has learned a relationship between a condition of a second patient, a medical practice performed on the second patient, and a change in condition of the second patient caused by the medical practice performed on the second patient.

### (Supplementary Note 17)

A medical planning assistance method performed by an information processing device, the medical planning assistance method including:
acquiring patient condition information regarding a condition of a first patient;
estimating a recommended medical practice for the first patient based on the acquired patient condition information and an estimation model; and
outputting the estimated recommended medical practice,
in which the estimation model is a model that has learned a relationship between a condition of the second patient, a medical practice performed on the second patient, and a change in condition of the second patient caused by the medical practice performed on the second patient.

### (Supplementary Note 18)

A recording medium storing a medical planning assistance program for causing a computer to execute:
an acquisition process of acquiring patient condition information regarding a condition of a first patient;
an estimation process of estimating a recommended medical practice for the first patient based on the patient condition information acquired by the acquisition process and an estimation model; and
an output process of outputting the recommended medical practice estimated by the estimation process,
in which the estimation model is a model that has learned a relationship between a condition of a second patient, a medical practice performed on the second patient, and a change in condition of the second patient caused by the medical practice performed on the second patient.

### REFERENCE SIGNS LIST

10 Medical planning assistance system
101 Patient condition information
102 Medical practice Information
103 Condition change information
104 Recommended medical practice
11 Acquisition unit
12 Classification unit
13 Analysis unit
14 Model generation unit
140 Estimation model
15 Estimation unit
16 Output unit
20 Management terminal device
200 Display screen
30 Medical device
40 Medical planning assistance system
400 Patient condition information
41 Acquisition unit
42 Estimation unit
420 Recommended medical practice
43 Estimation model
431 Condition of second patient
432 Medical practice performed on second patient
433 Change in condition of second patient
44 Output unit
900 Information processing system
901 CPU
902 ROM
903 RAM
904 Hard disk (storage device)
905 Communication interface
906 Bus
907 Recording medium
908 Reader/writer
909 Input/output interface

What is claimed is:

1. A medical planning assistance system comprising:
at least one memory storing a computer program; and
at least one processor configured to execute the computer program to
acquire patient condition information regarding a condition of a first patient;
estimate a recommended medical practice for the first patient based on the acquired patient condition information and an estimation model; and
output the estimated recommended medical practice,
wherein the estimation model is a model that has learned a relationship between a condition of a second patient, a medical practice performed on the second patient, and a change in condition of the second patient caused by the medical practice performed on the second patient.

2. The medical planning assistance system according to claim 1,
wherein the patient condition information for the first patient includes a history of biological conditions for the first patient and a history of medical practices performed on the first patient.

3. The medical planning assistance system according to claim 1,
wherein the condition of each of the first and second patients includes at least one of a body temperature, a heart rate, a blood pressure, a blood condition, a urine condition, a feces condition, an organ condition, a consciousness condition, a pain condition, and a joint movement condition.

4. The medical planning assistance system according to claim 1,
wherein the recommended medical practice and the medical practice performed on the first and second patients include at least one of surgery, medication treatment, radiation treatment, and rehabilitation treatment.

5. The medical planning assistance system according to claim 4,
wherein the surgery includes a body site on which the surgery is to be performed and a surgery method,
the medication treatment includes at least one of a type of medicine to be administered, an administration method, and a dosage,
the radiation treatment includes at least one of a type of radiation to be used, an irradiation location, and an irradiation time, and
the rehabilitation treatment includes at least one of a body site to be treated and a type of rehabilitation.

6. The medical planning assistance system according to claim 1, wherein the processor is configured to execute the computer program to
generate or update the estimation model based on the condition of the second patient, the medical practice performed on the second patient, and the change in condition of the second patient caused by the medical practice performed on the second patient.

7. The medical planning assistance system according to claim 6, wherein the processor is configured to execute the computer program to
estimate a medical practice recommended for the second patient based on the condition of the second patient, the medical practice performed on the second patient, and the estimation model,
when the change in condition of the second patient indicates that the condition of the second patient has been improved, update the estimation model to reduce a difference between the medical practice recommended for the second patient and the medical practice performed on the second patient, and
when the change in condition of the second patient indicates that the condition of the second patient has not been improved, update the estimation model to increase a difference between the medical practice recommended for the second patient and the medical practice performed on the second patient.

8. The medical planning assistance system according to claim 6, wherein the processor is configured to execute the computer program to
classify the medical practice performed on the second patient into a group based on similarity,
generate or update the estimation model based on the group and the change in condition of the second patient caused by the medical practice performed on the second patient, and
estimate the group to which the recommended medical practice belongs.

9. The medical planning assistance system according to claim 8, wherein the processor is configured to execute the computer program to
classify the medical practice performed on the second patient into the group having a hierarchical structure based on the similarity.

10. The medical planning assistance system according to claim 8, wherein the processor is configured to execute the computer program to
classify a first medical practice using a first medicine and a second medical practice using a second medicine into the same group, the second medicine having an equivalent effect to the first medicine while being different in type from the first medicine.

11. The medical planning assistance system according to claim 10,
wherein the first medicine is an original medicine, and the second medicine is a generic medicine of the first medicine.

12. The medical planning assistance system according to claim 6, wherein the processor is configured to execute the computer program to
analyze whether a combination of a plurality of medical practices performed on the second patient has affected the condition of the second patient,
specify the combination of medical practices which has affected the condition of the second patient,
use the specified combination of medical practices as the medical practice performed on the second patient, and
estimate the combination recommended for the first patient.

13. The medical planning assistance system according to claim 1, wherein the processor is configured to execute the computer program to
control a display device to display the recommended medical practice when an input operation is performed to input the patient condition information for the first patient to the estimation means.

14. The medical planning assistance system according to claim 13, wherein the processor is configured to execute the computer program to
control the display device to display information for prompting a doctor to perform an input operation indicating whether to approve the recommended medical practice.

15. The medical planning assistance system according to claim 14, wherein the processor is configured to execute the computer program to,
when the input operation performed by the doctor indicates that the recommended medical practice has been approved, generate control information for a medical device that performs the recommended medical practice, and output the generated control information to the medical device

16. (canceled)

17. A medical planning assistance method performed by an information processing device, the medical planning assistance method comprising:
acquiring patient condition information regarding a condition of a first patient;
estimating a recommended medical practice for the first patient based on the acquired patient condition information and an estimation model; and
outputting the estimated recommended medical practice,
wherein the estimation model is a model that has learned a relationship between a condition of the second patient, a medical practice performed on the second patient, and a change in condition of the second patient caused by the medical practice performed on the second patient.

18. A non-transitory computer-readable recording medium storing a medical planning assistance program for causing a computer to execute:
an acquisition process of acquiring patient condition information regarding a condition of a first patient;
an estimation process of estimating a recommended medical practice for the first patient based on the patient condition information acquired by the acquisition process and an estimation model; and
an output process of outputting the recommended medical practice estimated by the estimation process,
wherein the estimation model is a model that has learned a relationship between a condition of a second patient, a medical practice performed on the second patient, and a change in condition of the second patient caused by the medical practice performed on the second patient.
